(19) United States
(12) Patent Application Publication
(10) Pub. No.: US 2023/0401529 A1
(43) Pub. Date: Dec. 14, 2023

(54) SYSTEM AND METHOD FOR AUTOMATIC DETECTION FOR MULTIPLE FAILED ORDERS AT A BACK END PHARMACY

(71) Applicant: Express Scripts Strategic Development, Inc., St. Louis, MO (US)

(72) Inventor: Xiaojian Wang, St. Louis, MO (US)

(21) Appl. No.: 17/835,097

(22) Filed: Jun. 8, 2022

(51) Int. Cl. G06Q 10/08; B65G 1/137; G06Q 30/06; G06Q 10/04; G16H 20/10; G16H 40/20; G06N 20/00

(52) U.S. Cl. CPC G06Q 10/087; B65G 1/1373; G06Q 30/0635; G06Q 10/04; G16H 20/10; G16H 40/20; G06N 20/00

(57) ABSTRACT

A method for predicting pharmacy order failures includes receiving sensor data from one or more sensors associated with pharmacy order fulfilment, and identifying at least one pharmacy order package associated with the sensor data. The method also includes updating data associated with the at least one pharmacy order package based on at least some of the sensor data, and providing, to a predictive model, the updated data, The predictive model may be configured to predict one or more failures in a pharmacy order associated with the at least one pharmacy order package. The method also includes, in response to receiving at least one prediction output from the predictive model indicating at least one failure prediction, initiating at least one corrective action.

### TECHNICAL FIELD

This disclosure relates to pharmacy orders, and in particular to systems and methods for automatically detecting multiple failed orders at a back end pharmacy.

### BACKGROUND

Medications, such as prescription medications, over-the-counter medications, vitamins, supplements, and the like, are increasingly being delivered by a medication provider, such as a high-volume pharmacy and the like, to a residence or other location of an individual requiring such medications. Medications may be delivered using a variety of delivery services, such as a postal service, a parcel delivery service, a contractor, or other service under direct control of a corresponding medication provider, and the like. Further, such medications may be part of pharmacy order packaged at the pharmacy with other medications for a recipient, medication material (e.g., paperwork indicating mediation information, warnings, dosage, and the like), medication accessories (e.g., cleaning supplies, injection mechanisms, and the like), and so on.

### SUMMARY

This disclosure relates generally to pharmacy order fulfilment.

An aspect of the disclosed embodiments includes a system for predicting pharmacy order failures. The system includes a processor and a memory. The memory includes instructions that, when executed by the processor, cause the processor to: receive, in real-time, sensor data from one or more sensors associated with pharmacy order fulfilment of a high-volume pharmacy; identify at least one pharmacy order package associated with the sensor data; update, in a database, data associated with the at least one pharmacy order package based on at least some of the sensor data; provide, to a predictive model, the updated data associated with the at least one pharmacy order package, the predictive model being configured to predict one or more failures in a pharmacy order associated with the at least one pharmacy order package; and, in response to receiving at least one prediction output from the predictive model indicating at least one failure prediction in the pharmacy order associated with the at least one pharmacy order package, perform at least one corrective action. The at least one failure prediction may correspond to a contradiction between an expected value indicated by the updated data associated with the at least one pharmacy order package and a predicted actual value indicated by the updated data the at least one pharmacy order package.

Another aspect of the disclosed embodiments includes a method for predicting pharmacy order failures. The method includes receiving, in real-time, sensor data from one or more sensors associated with pharmacy order fulfilment of a high-volume pharmacy, and identifying at least one pharmacy order package associated with the sensor data. The method also includes updating, in a database, data associated with the at least one pharmacy order package based on at least some of the sensor data, and providing, to a predictive model, the updated data associated with the at least one pharmacy order package. The predictive model may be configured to predict one or more failures in a pharmacy order associated with the at least one pharmacy order package. The method also includes, in response to receiving at least one prediction output from the predictive model indicating at least one failure prediction in the pharmacy order associated with the at least one pharmacy order package, initiating at least one corrective action. The at least one failure prediction may correspond to a contradiction between an expected value indicated by the updated data associated with the at least one pharmacy order package and a predicted actual value indicated by the updated data the at least one pharmacy order package.

Another aspect of the disclosed embodiments includes a system for predicting pharmacy order failures. The system includes a processor and a memory. The memory includes instructions that, when executed by the processor, cause the processor to: receive, in real-time, sensor data from one or more weight sensors associated with pharmacy order fulfilment of a high-volume pharmacy; identify at least one pharmacy order package associated with the sensor data; update, in a database, data associated with the at least one pharmacy order package based on at least some of the sensor data; provide, to an artificial intelligence engine configured to execute at least one machine learning model that is initially trained using at least historical pharmacy order package data, the updated data associated with the at least one pharmacy order package, the at least one machine learning model being configured to predict one or more failures in a pharmacy order associated with the at least one pharmacy order package; and, in response to receiving at least one prediction output from the predictive model indicating at least one failure prediction in the pharmacy order associated with the at least one pharmacy order package, perform at least one corrective action. The at least one failure prediction may correspond to a contradiction between an expected weight value indicated by the updated data associated with the at least one pharmacy order package and a predicted actual weight value indicated by the updated data the at least one pharmacy order package.

These and other aspects of the present disclosure are disclosed in the following detailed description of the embodiments, the appended claims, and the accompanying figures.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1A generally illustrates a functional block diagram of a system including a high-volume pharmacy according to the principles of the present disclosure.

FIG. 1B generally illustrates a computing device according to the principles of the present disclosure.

FIG. 2 generally illustrates a functional block diagram of a pharmacy fulfillment device, which may be deployed within the system of FIG. 1A.

FIG. 3 generally illustrates a functional block diagram of an order processing device, which may be deployed within the system of FIG. 1A.

FIG. 4 generally illustrates a pharmacy order failure prediction method according to the principles of the present disclosure.

### DETAILED DESCRIPTION

The following discussion is directed to various embodiments of the present disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

As described, medications, such as prescription medications, over-the-counter medications, vitamins, supplements, and the like, are increasingly being delivered by a medication provider, such as a high-volume pharmacy and the like, to a residence or other location of an individual requiring such medications. Medications may be delivered using a variety of delivery services, such as a postal service, a parcel delivery service, a contractor, or other service under direct control of a corresponding medication provider, and the like. Further, such medications may be part of pharmacy order packaged at the pharmacy with other medications for a recipient, medication material (e.g., paperwork indicating mediation information, warnings, dosage, and the like), medication accessories (e.g., cleaning supplies, injection mechanisms, and the like), and so on.

The high-volume pharmacy may employ on order packaging process that includes using one or more manual or automated order packaging mechanisms. For example, the high-volume pharmacy may include an automated medication dispenser that dispenses medications into a medication container (e.g., a bottle, and the like) according to information associated with the order (e.g., such as mediation quality, and the like), packaging weigh stations, and the like. Occasionally, an order may fail during the order packaging process. For example, medication may be dispensed into a medication container and transferred to an associated order package. The order package may be weight to verify the contents of the package. If the package does not weight the same as an expected weight, the order may be flagged as a failure. It should be understood that the examples described herein with respect to the packaging processes of the high-volume pharmacy are illustrative and the packaging processes may include additional or other features including or instead of those described herein.

Typically, procedures for responding to such failed orders do not include real-time data for any failed orders and failure and/or other order data may not be stored in a central location (e.g., for access by operators of the high-volume pharmacy). Instead, manual adjusts to, for example, weight information, may be made based on individual experience and/or expertise. For example, a first team of individuals may rely on an information technology team to manually load data to a storage device and then download the data. The first team may then generate or update an existing spreadsheet to indicate the data, which may already be stale (e.g., more than several days old). A second team may receive a report from the first team based on the data. The second team may manually search the data for failed orders. As such, adjustments made in response to the failures may take place days or weeks after the first indication of a failure. During this time, other failures may occur that may have been avoidable had the adjustments been made earlier.

Accordingly, systems and methods, such as those described herein, configured to predict one or more pharmacy order failures, may be desirable. In some embodiments, the systems and methods described herein may be configured to reduce or eliminate order failure analysis delays. The systems and methods described herein may be configured to capture and/or store order packaging data in real-time or near real-time (e.g., in a relatively short period, such as seconds, microseconds, and the like). The systems and methods described herein may be configured to store data in a centralized data structure, such as on a distributed database or in a database in a single location.

The systems and methods described herein may be configured to automatically detect failed orders and/or automatically join package information (e.g., such as weight of the package, standard weight, vial information, and/or the like), which may reduce or eliminate the need or an individual to look up that information in one more data stores or applications. The systems and methods described herein may be configured to provide one or more predictive models applied to the data. The one or more predictive models may automatically detect failed order and/or may change information the database (e.g., without any human effort).

The systems and methods described herein may be configured to provide the real-time data (e.g., which reduce or eliminate a lagging period). The systems and methods described herein may be configured to reduce or eliminate manual effort in retrieving the data. The systems and methods described herein may be configured to provide more useful data. The systems and methods described herein may be configured to provide visualization that allows an end user to validate orders. The systems and methods described herein may be configured to provide one or more predictive models that detect changes in expected values (e.g., weight values and the like) associated with pharmacy order packages, which may avoid generating false alarms.

In some embodiments, the systems and methods described herein may be configured to receive, in real-time, sensor data from one or more sensors associated with pharmacy order fulfilment of a high-volume pharmacy. The one or more sensors includes at least one or more weight sensors, other suitable sensors, or a combination thereof. The systems and methods described herein may be configured to identify at least one pharmacy order package associated with the sensor data. The systems and methods described herein may be configured to update, in a database, data associated with the at least one pharmacy order package based on at least some of the sensor data.

The systems and methods described herein may be configured to provide, to a predictive model, the updated data associated with the at least one pharmacy order package. The predictive model may be configured to predict one or more failures in a pharmacy order associated with the at least one pharmacy order package. The predictive model may include at least one machine learning model executed by an artificial intelligence engine. The at least one machine learning model may be initially trained using at least historical pharmacy order package data. The at least one machine learning model may be subsequently trained using at least output generated by the at least one machine learning model.

The systems and methods described herein may be configured to, in response to receiving at least one prediction output from the predictive model indicating at least one failure prediction in the pharmacy order associated with the at least one pharmacy order package, perform at least one corrective action. The at least one failure prediction may correspond to a contradiction between an expected value indicated by the updated data associated with the at least one pharmacy order package and a predicted actual value indicated by the updated data the at least one pharmacy order package. For example, the expected value may correspond to an expected weight of at least one component of the at least one pharmacy order package and the predicted actual value may correspond to a predicted actual weight generated by the predictive model based on data associated with one or more other pharmacy order packages.

The systems and methods described herein may be configured to perform the act least one corrective action by determining whether the predicted actual value indicates a change in the expected value. The systems and methods described herein may be configured to, in response to a determination that the predicted actual value indicates a change in the expected value, update, based on the predicted actual value, the expected value associated with the at least one pharmacy order package and one or more other pharmacy order packages having an expected value corresponding to the expected value of the at least one pharmacy order package. The systems and methods described herein may be configured to, in response to a determination that the predicted actual value does not indicate a change in the expected value, generate, for display, an output indicating the at least one failure prediction. The systems and methods described herein may be configured to provide, at a display, the output indicating the at least one failure prediction.

The systems and methods described herein may be configured to, in response to the determination that the predicted actual value does not indicate a change in the expected value, preemptively change one or more aspects of the at least one pharmacy order package responsive to the predicted actual value.

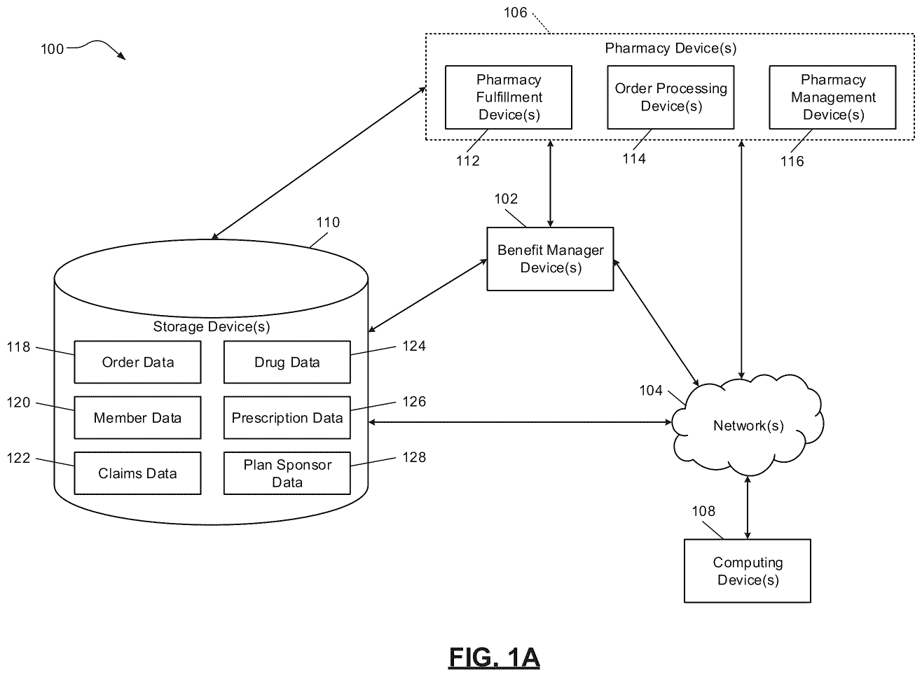

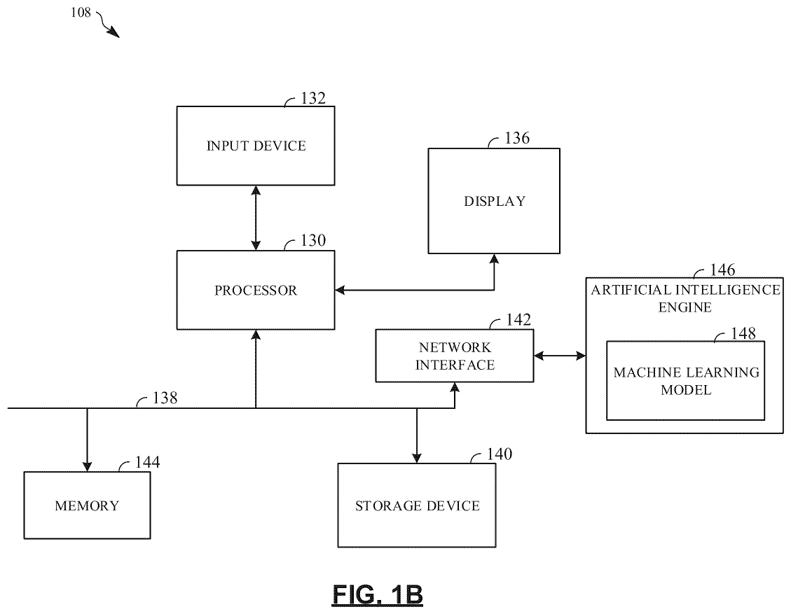

FIG. 1A is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104. The system 100 may also include a storage device 110.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in the storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 100) following performance of at least some of the aforementioned operations.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Virginia.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfilment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the use of the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

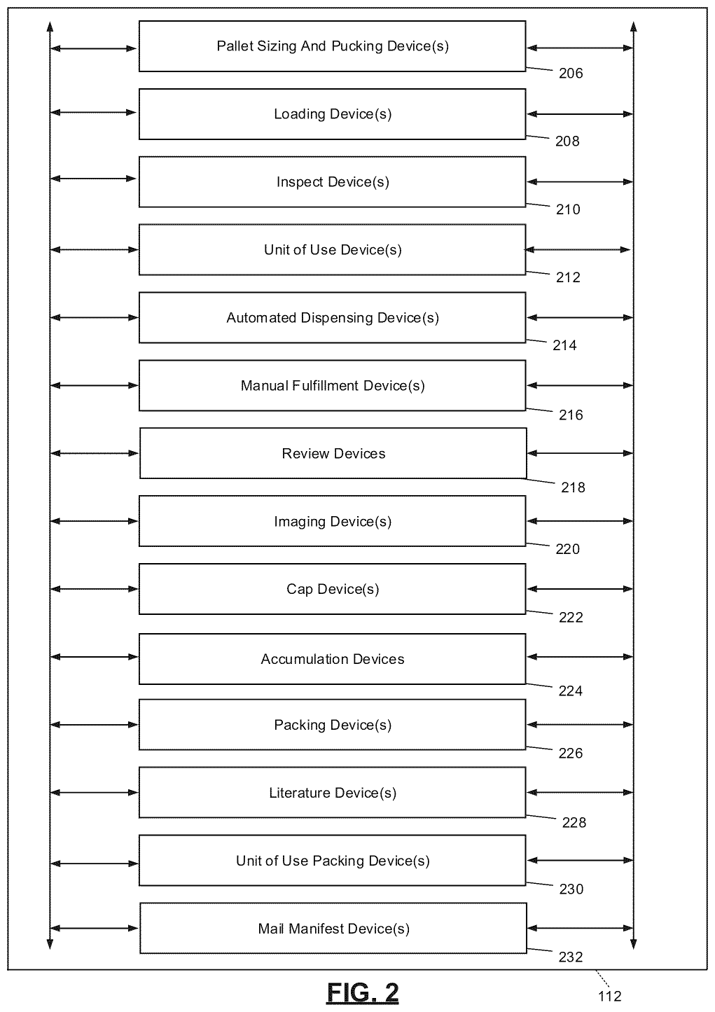

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

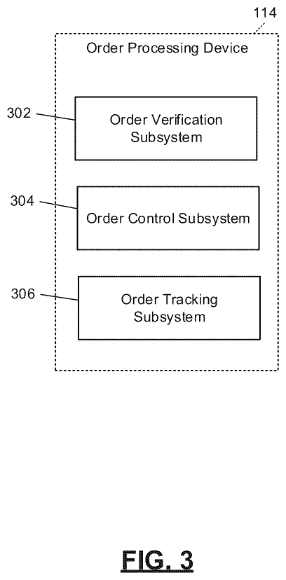

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may include order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, and the like. The order tracking subsystem 306 may store data locally (for example, in a memory) or as a portion of the order data 118 stored in the storage device 110.

In some embodiments, the system 100 may include one or more computing devices 108, as is generally illustrated in FIG. 1B. The computing device 108 may include any suitable computing device, such as a mobile computing device, a desktop computing device, a laptop computing device, a server computing device, other suitable computing device, or a combination thereof. The computing device 108 may be used by a user accessing the pharmacy associated with the system 100, as described. Additionally, or alternatively, the computing device 108 may be configured to identify an optimum or substantially optimum combination of data objects, as described.

The computing device 108 may include a processor 130 configured to control the overall operation of computing device 108. The processor 130 may include any suitable processor, such as those described herein. The computing device 108 may also include a user input device 132 that is configured to receive input from a user of the computing device 108 and to communicate signals representing the input received from the user to the processor 130. For example, the user input device 132 may include a button, keypad, dial, touch screen, audio input interface, visual/image capture input interface, input in the form of sensor data, etc.

The computing device 108 may include a display 136 that may be controlled by the processor 130 to display information to the user. A data bus 138 may be configured to facilitate data transfer between, at least, a storage device 140 and the processor 130. The computing device 108 may also include a network interface 142 configured to couple or connect the computing device 108 to various other computing devices or network devices via a network connection, such as a wired or wireless connection, such as the network 104. In some embodiments, the network interface 142 includes a wireless transceiver.

The storage device 140 may include a single disk or a plurality of disks (e.g., hard drives), one or more solid-state drives, one or more hybrid hard drives, and the like. The storage device 140 may include a storage management module that manages one or more partitions within the storage device 140. In some embodiments, storage device 140 may include flash memory, semiconductor (solid state) memory or the like. The computing device 108 may also include a memory 144. The memory 144 may include Random Access Memory (RAM), a Read-Only Memory (ROM), or a combination thereof. The memory 144 may store programs, utilities, or processes to be executed in by the processor 130. The memory 144 may provide volatile data storage, and stores instructions related to the operation of the computing device 108. In some embodiments, the processor 130 may be configured to execute instructions stored on the memory 144 to, at least, perform various aspects of the systems and methods described herein.

In some embodiments, the computing device 108 may include an artificial intelligence engine 146 configured to use one or more machine learning models 148 configured to perform at least some aspects of the systems and methods described herein. The artificial intelligence engine 146 may include any suitable artificial intelligence engine and may be disposed on computing device 108 or remotely located from the computing device 108, such as in a cloud computing device or other suitable remotely located computing device. The computing device 108 may include a training engine capable of generating the one or more machine learning models 148. The one or more machine learning models 148 may be trained using any suitable data, including those described herein. Additionally, or alternatively, the one or more machine learning models 148 may be iteratively trained (e.g., subsequent to an initiate training) using output from the one or more machine learning models 148 (e.g., as feedback, which may or may not include input from a user indicating accuracy of one or more predictions associated with the output of the one or more machine learning models 148).

In some embodiments, the computing device 108 may be configured to predict one or more pharmacy order failures. For example, the computing device 108 may receive, in real-time, sensor data from one or more sensors associated with pharmacy order fulfilment of the high-volume pharmacy. The one or more sensors includes at least one or more weight sensors, other suitable sensors, or a combination thereof. The computing device 108 may identify at least one pharmacy order package associated with the sensor data. The s computing device 108 may update, in a database (e.g., which may include or be stored on the storage device 110 or other suitable database or location), data associated with the at least one pharmacy order package based on at least some of the sensor data.

The computing device 108 may provide, to a predictive model, the updated data associated with the at least one pharmacy order package. The predictive model may be configured to predict one or more failures in a pharmacy order associated with the at least one pharmacy order package. The predictive model may include at least one machine learning model, such as the machine learning model 148 or other suitable machine learning model, executed by an artificial intelligence engine, such as the artificial intelligence engine 146 or other suitable artificial intelligence engine. The machine learning model 148 may be initially trained using at least historical pharmacy order package data and/or subsequently trained using at least output generated by the machine learning model 148.

The computing device 108 may, in response to receiving at least one prediction output from the predictive model indicating at least one failure prediction in the pharmacy order associated with the at least one pharmacy order package, perform at least one corrective action. The at least one failure prediction may correspond to a contradiction between an expected value indicated by the updated data associated with the at least one pharmacy order package and a predicted actual value indicated by the updated data the at least one pharmacy order package. For example, the expected value may correspond to an expected weight of at least one component of the at least one pharmacy order package and the predicted actual value may correspond to a predicted actual weight generated by the predictive model based on data associated with one or more other pharmacy order packages.

The computing device 108 may perform the act least one corrective action by determining whether the predicted actual value indicates a change in the expected value. The computing device 108 may, in response to a determination that the predicted actual value indicates a change in the expected value, update, based on the predicted actual value, the expected value associated with the at least one pharmacy order package and one or more other pharmacy order packages having an expected value corresponding to the expected value of the at least one pharmacy order package. For example, the computing device 1-08 may determine that the predicted actual weight value corresponds to a revised expected eight for a given medication (e.g., revised based on a modification to the medication from the medication manufacturer). The computing device 1-08 may adjust the expected value of the medication to correspond to the predicted actual value, which may prevent future pharmacy order packing failures. The computing device 108 may, in response to a determination that the predicted actual value does not indicate a change in the expected value, generate, for display, an output indicating the at least one failure prediction. The systems and methods described herein may be configured to provide, at a display (e.g., such as the display 136 or other suitable display), the output indicating the at least one failure prediction.

The computing device 108 may, in response to the determination that the predicted actual value does not indicate a change in the expected value, preemptively change one or more aspects of the at least one pharmacy order package responsive to the predicted actual value. For example, the computing device 108 may adjust information used by an automated dispensing mechanism, such that the automated dispensing mechanism dispenses an amount of medication corresponding to the expected weight value.

In some embodiments, the computing device 108 and/or the system 100 may perform the methods described herein. However, the methods described herein as performed by the computing device 108 and/or the system 100 are not meant to be limiting, and any type of software executed on a computing device or a combination of various computing devices can perform the methods described herein without departing from the scope of this disclosure.

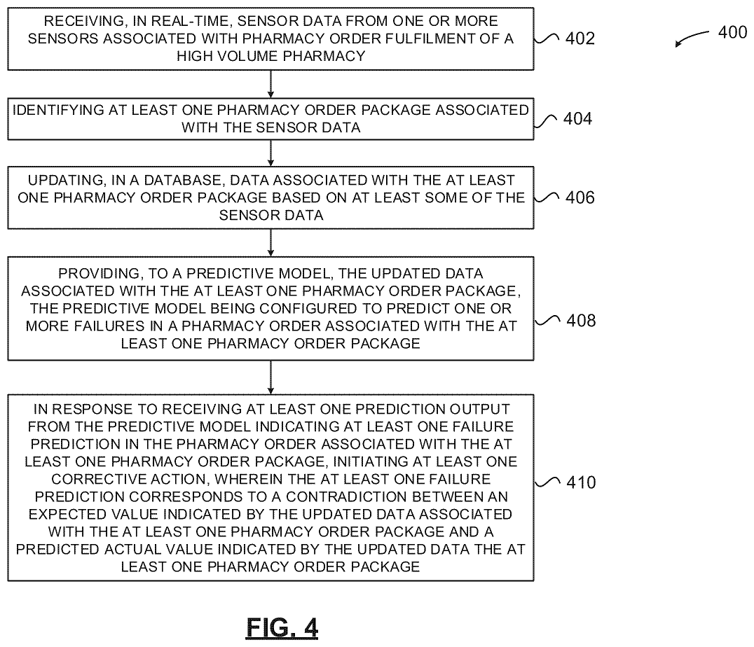

FIG. 4 is a flow diagram generally illustrating a pharmacy order failure prediction method 400 according to the principles of the present disclosure. At 402, the method 400 receives, in real-time, sensor data from one or more sensors associated with pharmacy order fulfilment of a high-volume pharmacy. For example, the computing device 108 may receive, in real-time, the sensor data from the one or more sensors associated with the pharmacy order fulfilment of the high-volume pharmacy.

At 404, the method 400 identifies at least one pharmacy order package associated with the sensor data. For example, the computing device 108 may identify the at least one pharmacy order package associated with the sensor data.

At 406, the method 400 updates, in a database, data associated with the at least one pharmacy order package based on at least some of the sensor data. For example, the computing device 108 may update, in the database (e.g., which may include or be disposed on the storage device 110), the data associated with the at least one pharmacy order package based on at least some of the sensor data.

At 408, the method 400 provides, to a predictive model, the updated data associated with the at least one pharmacy order package. The predictive model may be configured to predict one or more failures in a pharmacy order associated with the at least one pharmacy order package. For example, the computing device 108 may provide, to the machine learning model 148, the updated data associated with the at least one pharmacy order package. The machine learning model may be configured to predict one or more failures in a pharmacy order associated with the at least one pharmacy order package

At 410, the method 400, in response to receiving at least one prediction output from the predictive model indicating at least one failure prediction in the pharmacy order associated with the at least one pharmacy order package, initiates at least one corrective action. For example, the computing device 108 may, in response to receiving the at least one prediction output from the predictive model indicating the at least one failure prediction in the pharmacy order associated with the at least one pharmacy order package, initiate at least one corrective action. The at least one failure prediction may correspond to a contradiction between an expected value indicated by the updated data associated with the at least one pharmacy order package and a predicted actual value indicated by the updated data the at least one pharmacy order package.

In some embodiments, a system for predicting pharmacy order failures includes a processor and a memory. The memory includes instructions that, when executed by the processor, cause the processor to: receive, in real-time, sensor data from one or more sensors associated with pharmacy order fulfilment of a high-volume pharmacy; identify at least one pharmacy order package associated with the sensor data; update, in a database, data associated with the at least one pharmacy order package based on at least some of the sensor data; provide, to a predictive model, the updated data associated with the at least one pharmacy order package, the predictive model being configured to predict one or more failures in a pharmacy order associated with the at least one pharmacy order package; and, in response to receiving at least one prediction output from the predictive model indicating at least one failure prediction in the pharmacy order associated with the at least one pharmacy order package, perform at least one corrective action. The at least one failure prediction may correspond to a contradiction between an expected value indicated by the updated data associated with the at least one pharmacy order package and a predicted actual value indicated by the updated data the at least one pharmacy order package.

In some embodiments, the one or more sensors includes at least a weight sensor. In some embodiments, the expected value corresponds to an expected weight of at least one component of the at least one pharmacy order package and the predicted actual value corresponds to a predicted actual weight generated by the predictive model based on data associated with one or more other pharmacy order packages. In some embodiments, the instructions further causing the processor to perform the act least one corrective action by determining whether the predicted actual value indicates a change in the expected value, and, in response to a determination that the predicted actual value indicates a change in the expected value, updating, based on the predicted actual value, the expected value associated with the at least one pharmacy order package and one or more other pharmacy order packages having an expected value corresponding to the expected value of the at least one pharmacy order package. In some embodiments, the instructions further causing the processor to perform the act least one corrective action further by, in response to a determination that the predicted actual value does not indicate a change in the expected value, generating, for display, an output indicating the at least one failure prediction, and providing, at a display, the output indicating the at least one failure prediction. In some embodiments, the instructions further causing the processor to perform the act least one corrective action further by, in response to the determination that the predicted actual value does not indicate a change in the expected value, preemptively changing one or more aspects of the at least one pharmacy order package responsive to the predicted actual value. In some embodiments, the predictive model includes at least one machine learning model executed by an artificial intelligence engine. In some embodiments, the at least one machine learning model is initially trained using at least historical pharmacy order package data. In some embodiments, the at least one machine learning model is subsequently trained using at least output generated by the at least one machine learning model.

In some embodiments, a method for predicting pharmacy order failures includes receiving, in real-time, sensor data from one or more sensors associated with pharmacy order fulfilment of a high-volume pharmacy, and identifying at least one pharmacy order package associated with the sensor data. The method also includes updating, in a database, data associated with the at least one pharmacy order package based on at least some of the sensor data, and providing, to a predictive model, the updated data associated with the at least one pharmacy order package. The predictive model may be configured to predict one or more failures in a pharmacy order associated with the at least one pharmacy order package. The method also includes, in response to receiving at least one prediction output from the predictive model indicating at least one failure prediction in the pharmacy order associated with the at least one pharmacy order package, initiating at least one corrective action. The at least one failure prediction may correspond to a contradiction between an expected value indicated by the updated data associated with the at least one pharmacy order package and a predicted actual value indicated by the updated data the at least one pharmacy order package.

In some embodiments, the one or more sensors includes at least a weight sensor. In some embodiments, the expected value corresponds to an expected weight of at least one component of the at least one pharmacy order package and the predicted actual value corresponds to a predicted actual weight generated by the predictive model based on data associated with one or more other pharmacy order packages. In some embodiments, the at least one corrective active includes determining whether the predicted actual value indicates a change in the expected value, and, in response to a determination that the predicted actual value indicates a change in the expected value, updating, based on the predicted actual value, the expected value associated with the at least one pharmacy order package and one or more other pharmacy order packages having an expected value corresponding to the expected value of the at least one pharmacy order package. In some embodiments, the at least one corrective action further includes, in response to a determination that the predicted actual value does not indicate a change in the expected value, generating, for display, an output indicating the at least one failure prediction, and providing, at a display, the output indicating the at least one failure prediction. In some embodiments, the at least one corrective action further includes, in response to the determination that the predicted actual value does not indicate a change in the expected value, preemptively changing one or more aspects of the at least one pharmacy order package responsive to the predicted actual value. In some embodiments, the predictive model includes at least one machine learning model executed by an artificial intelligence engine. In some embodiments, the at least one machine learning model is initially trained using at least historical pharmacy order package data. In some embodiments, the at least one machine learning model is subsequently trained using at least output generated by the at least one machine learning model.

In some embodiments, a system for predicting pharmacy order failures includes a processor and a memory. The memory includes instructions that, when executed by the processor, cause the processor to: receive, in real-time, sensor data from one or more weight sensors associated with pharmacy order fulfilment of a high-volume pharmacy; identify at least one pharmacy order package associated with the sensor data; update, in a database, data associated with the at least one pharmacy order package based on at least some of the sensor data; provide, to an artificial intelligence engine configured to execute at least one machine learning model that is initially trained using at least historical pharmacy order package data, the updated data associated with the at least one pharmacy order package, the at least one machine learning model being configured to predict one or more failures in a pharmacy order associated with the at least one pharmacy order package; and, in response to receiving at least one prediction output from the predictive model indicating at least one failure prediction in the pharmacy order associated with the at least one pharmacy order package, perform at least one corrective action. The at least one failure prediction may correspond to a contradiction between an expected weight value indicated by the updated data associated with the at least one pharmacy order package and a predicted actual weight value indicated by the updated data the at least one pharmacy order package.

In some embodiments, the at least one machine learning model is subsequently trained using at least output generated by the at least one machine learning model.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Implementations of the systems, algorithms, methods, instructions, etc., described herein may be realized in hardware, software, or any combination thereof. The hardware may include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. In the claims, the term “processor” should be understood as encompassing any of the foregoing hardware, either singly or in combination. The terms “signal” and “data” are used interchangeably.

What is claimed is:

1. A system for predicting pharmacy order failures, the system comprising:
a processor; and
a memory including instructions that, when executed by the processor, cause the processor to:
receive, in real-time, sensor data from one or more sensors associated with pharmacy order fulfilment of a high-volume pharmacy;
identify at least one pharmacy order package associated with the sensor data;
update, in a database, data associated with the at least one pharmacy order package based on at least some of the sensor data;
provide, to a predictive model, the updated data associated with the at least one pharmacy order package, the predictive model being configured to predict one or more failures in a pharmacy order associated with the at least one pharmacy order package; and
in response to receiving at least one prediction output from the predictive model indicating at least one failure prediction in the pharmacy order associated with the at least one pharmacy order package, perform at least one corrective action, wherein the at least one failure prediction corresponds to a contradiction between an expected value indicated by the updated data associated with the at least one pharmacy order package and a predicted actual value indicated by the updated data the at least one pharmacy order package.

2. The system of claim 1, wherein the one or more sensors includes at least a weight sensor.

3. The system of claim 2, wherein the expected value corresponds to an expected weight of at least one component of the at least one pharmacy order package and the predicted actual value corresponds to a predicted actual weight generated by the predictive model based on data associated with one or more other pharmacy order packages.

4. The system of claim 1, wherein the instructions further causing the processor to perform the act least one corrective action by:
determining whether the predicted actual value indicates a change in the expected value; and
in response to a determination that the predicted actual value indicates a change in the expected value, updating, based on the predicted actual value, the expected value associated with the at least one pharmacy order package and one or more other pharmacy order packages having an expected value corresponding to the expected value of the at least one pharmacy order package.

5. The system of claim 4, wherein the instructions further causing the processor to perform the act least one corrective action further by:
in response to a determination that the predicted actual value does not indicate a change in the expected value, generating, for display, an output indicating the at least one failure prediction; and
providing, at a display, the output indicating the at least one failure prediction.

6. The system of claim 5, wherein the instructions further causing the processor to perform the act least one corrective action further by, in response to the determination that the predicted actual value does not indicate a change in the expected value, preemptively changing one or more aspects of the at least one pharmacy order package responsive to the predicted actual value.

7. The system of claim 1, wherein the predictive model includes at least one machine learning model executed by an artificial intelligence engine.

8. The system of claim 7, wherein the at least one machine learning model is initially trained using at least historical pharmacy order package data.

9. The system of claim 8, wherein the at least one machine learning model is subsequently trained using at least output generated by the at least one machine learning model.

10. A method for predicting pharmacy order failures, the method comprising:
receiving, in real-time, sensor data from one or more sensors associated with pharmacy order fulfilment of a high-volume pharmacy;
identifying at least one pharmacy order package associated with the sensor data;
updating, in a database, data associated with the at least one pharmacy order package based on at least some of the sensor data;
providing, to a predictive model, the updated data associated with the at least one pharmacy order package, the predictive model being configured to predict one or more failures in a pharmacy order associated with the at least one pharmacy order package; and
in response to receiving at least one prediction output from the predictive model indicating at least one failure prediction in the pharmacy order associated with the at least one pharmacy order package, initiating at least one corrective action, wherein the at least one failure prediction corresponds to a contradiction between an expected value indicated by the updated data associated with the at least one pharmacy order package and a predicted actual value indicated by the updated data the at least one pharmacy order package.

11. The method of claim 10, wherein the one or more sensors includes at least a weight sensor.

12. The method of claim 11, wherein the expected value corresponds to an expected weight of at least one component of the at least one pharmacy order package and the predicted actual value corresponds to a predicted actual weight generated by the predictive model based on data associated with one or more other pharmacy order packages.

13. The method of claim 10, wherein the at least one corrective active includes:
determining whether the predicted actual value indicates a change in the expected value; and
in response to a determination that the predicted actual value indicates a change in the expected value, updating, based on the predicted actual value, the expected value associated with the at least one pharmacy order package and one or more other pharmacy order packages having an expected value corresponding to the expected value of the at least one pharmacy order package.

14. The method of claim 13, wherein the at least one corrective action further includes:
in response to a determination that the predicted actual value does not indicate a change in the expected value, generating, for display, an output indicating the at least one failure prediction; and
providing, at a display, the output indicating the at least one failure prediction.

15. The method of claim 14, wherein the at least one corrective action further includes, in response to the determination that the predicted actual value does not indicate a change in the expected value, preemptively changing one or more aspects of the at least one pharmacy order package responsive to the predicted actual value.

16. The method of claim 10, wherein the predictive model includes at least one machine learning model executed by an artificial intelligence engine.

17. The method of claim 16, wherein the at least one machine learning model is initially trained using at least historical pharmacy order package data.

18. The method of claim 17, wherein the at least one machine learning model is subsequently trained using at least output generated by the at least one machine learning model.

19. A system for predicting pharmacy order failures, the system comprising:
a processor; and
a memory including instructions that, when executed by the processor, cause the processor to:
receive, in real-time, sensor data from one or more weight sensors associated with pharmacy order fulfilment of a high-volume pharmacy;
identify at least one pharmacy order package associated with the sensor data;
update, in a database, data associated with the at least one pharmacy order package based on at least some of the sensor data;
provide, to an artificial intelligence engine configured to execute at least one machine learning model that is initially trained using at least historical pharmacy order package data, the updated data associated with the at least one pharmacy order package, the at least one machine learning model being configured to predict one or more failures in a pharmacy order associated with the at least one pharmacy order package; and
in response to receiving at least one prediction output from the predictive model indicating at least one failure prediction in the pharmacy order associated with the at least one pharmacy order package, perform at least one corrective action, wherein the at least one failure prediction corresponds to a contradiction between an expected weight value indicated by the updated data associated with the at least one pharmacy order package and a predicted actual weight value indicated by the updated data the at least one pharmacy order package.

20. The system of claim 19, wherein the at least one machine learning model is subsequently trained using at least output generated by the at least one machine learning model.
